(19) United States
(12) Patent Application Publication
(10) Pub. No.: US 2015/0150868 A1
(43) Pub. Date: Jun. 4, 2015

(54) ARIPIPRAZOLE-ORGANIC ACID COCRYSTAL, PREPARATION OR COMPOSITION CONTAINING SAME, AND PREPARATION METHOD THEREFOR

(71) Applicant: GCB CO., LTD, Suwon-si, Gyeonggi-do (KR)

(72) Inventor: Shinbyoung Ahn, Ansan-si (KR)

(73) Assignee: GCB CO., LTD, Suwon-si, Gyeonggi-do (KR)

(21) Appl. No.: 14/408,554

(22) PCT Filed: Jun. 25, 2013
(86) PCT No.: PCT/KR2013/005574

§ 371 Date: Dec. 16, 2014

(30) Foreign Application Priority Data
Jun. 26, 2012 (KR) 10-2012-0068343

(51) Int. Cl. A61K 31/496; A61K 31/095; A61K 31/455; A61K 31/60; A61K 31/194

(52) U.S. Cl. CPC A61K 31/496; A61K 31/60; A61K 31/194; A61K 31/455; A61K 31/095

(57) ABSTRACT

According to the present invention, a formulation or composition containing the same, and a preparation method thereof, a novel cocrystal free of hygroscopicity can be obtained by adding an organic acid such as salicylic acid to an anhydrous aripiprazole crystal. In addition, an industrially useful grinding process is provided, which enables the convenience of preparation of the cocrystal and worker's safety to be ensured and allows the preparation of the cocrystal to be performed without needing a special chemical system.

### TECHNICAL FIELD

The present invention relates to an aripiprazole-organic acid cocrystal, a formulation or composition containing the same, and a preparation method thereof, and more particularly, to an aripiprazole-organic acid cocrystal consisting of aripiprazole and organic acid for medical use, a formulation or composition containing the same, and a preparation method thereof.

### BACKGROUND ART

Aripiprazole 7-{4-[4-(2,3-dichlorophenyl)-1-piperazinyl]-butoxy}-3,4-dihydrocarbostyril or 7-{4-[4-(2,3-dichlorophenyl)-1-piperazinyl]-butoxy}-3,4-dihydro-2(1H)-quinolinone, which has a molecular formula of C₂₃H₂₇Cl₂N₃O₂ (molecular weight: 448.38), is atypical antipsychotic drug useful for the treatment of schizophrenia. It is a second-generation atypical antipsychotic drug that has been marketed recently and has started to attract attention. It is available as 5, 10, 15, 20 and 30 mg tablets under the trade name of Abilify, and is also available as orally fast-disintegrating tablets or liquid formulations.

Regarding crystalline forms of aripiprazole, pharmaceutically acceptable salts of aripiprazole with organic acids (U.S. Pat. Nos. 4,734,416 and 5,006,528) were developed, and various crystalline forms such as low-hygroscopic polymorphic crystals (Korean Patent No. 10-0530731), hydrates, and solvates with methanol or the like (WO 2006/079548 A1, WO 2006/077584 A2, Limor Tessler and Israel Goldberg, Journal of Inclusion Phenomena and Macrocyclic Chemistry, 55, pp. 255-261, 2006) were reported.

Regarding cocrystal forms of aripiprazole, a cocrystal of aripiprazole and fumaric acid was recently developed (US Patent Publication No. 2009/0054455 A1). This cocrystal is not clearly distinguished from fumarate, and for this distinction, three-dimensional structural analysis is required. As used herein, the term “cocrystal” refers to a single distinct crystal formed by the interaction between two or more molecules while each of the molecules exists as a neutral molecule without chemical modification. A cocrystal differs from a salt formed by ionization of molecules, and also differs in terms of intermolecular interaction. In addition, a cocrystal differs from a salt or mixture consisting of the same components with respect to melting point, solubility, elution, hygroscopicity, chemical stability and the like, and the properties thereof vary depending on the kind of organic acid (coformer) coexisting therewith in drugs. Thus, the physical and pharmaceutical properties of cocrystals vary depending on the components thereof.

The bioavailability of an oral formulation is measured by the amount of the drug absorbed in the blood through the circulatory system after administration. Thus, it can be seen that the bioavailability of the drug is determined by the dissolution or release of the drug in the gastrointestinal tract and the systemic absorption of the released drug. The dissolution of a drug is closely associated with the solubility of the drug in a given system, that is, the gastrointestinal tract, and an increase in drug absorption leads to an increase in the bioavailability of the drug. If a drug is a sustained-release formulation, the number of daily drug administration can be reduced.

This drug improvement can be achieved not only by changing the composition of the formulation, but also by changing the properties or compositions of drug components to change the physical properties of the drug. This change in drug properties may be applied not only to oral formulations, but also to various other formulations, including ointments, patches and inhalation formulations.

However, in the prior art, there was a limit to increasing the utility of aripiprazole cocrystal structures.

### DISCLOSURE

### Technical Problem

The present invention has been made in order to solve the above-described problems occurring in the prior art, and it is an object of the present invention to provide a novel aripiprazole-organic acid cocrystal.

Another object of the present invention is to change the physical properties of aripiprazole by preparing the novel aripiprazole cocrystal as described above, and to increase the utility of aripiprazole by preparing a highly useful composition or formulation using the novel aripiprazole cocrystal. In the present invention, the cocrystal was prepared by a solvent-based crystallization process or an industrially more useful grinding process, and the structure thereof was confirmed by various instrumental analysis techniques. Specifically, in the present invention, novel five cocrystals, each consisting of aripiprazole and an organic acid, were prepared, and the structures thereof were analyzed. Among the prepared cocrystals, a single crystal of an aripiprazole-salicylic acid (1:1) cocrystal was prepared and analyzed by three-dimensional X-ray diffraction to confirm the actual structure of the cocrystal.

### Technical Solution

In order to achieve the object, an aripiprazole-organic acid cocrystal comprises aripiprazole and an organic acid.

Furthermore, the organic acid is one or more of salicylic acid, adipic acid, benzenesulfonic acid, nicotinic acid, and terephthalic acid, and a molecular ratio of the aripiprazole to the organic acid is 1:1 or 2:1.

In order to achieve the object, a method for preparing an aripiprazole-organic acid cocrystal, comprises forming a cocrystal from aripiprazole and an organic acid by a solvent process and a grinding process.

Furthermore, the organic acid is one or more of salicylic acid, adipic acid, benzenesulfonic acid, nicotinic acid, and terephthalic acid, and a molecular ratio of the aripiprazole to the organic acid is 1:1 or 2:1.

Furthermore, the method comprises the steps of: dissolving the aripiprazole and the organic acid in a solvent to form a solution; and either crystallizing the solution under cold conditions at a temperature of 0˜10° C., or evaporating the solvent from the solution, thereby obtaining the cocrystal, wherein the solvent is one or a mixture of two or more selected from a group consisting of aprotic solvents, dimethylformamide, dimethyl sulfoxide, dimethylacetamide, acetonitrile, ether-based solvents, tetrahydrofuran, 1,4-dioxane, alcohol-based solvents, methanol, ethanol, isopropanol, propanol, isobutanol, n-butanol, t-butanol, ethylene glycol, dichloromethane, acetone, methyl ethyl ketone, and distilled water.

Furthermore, the method comprises grinding the aripiprazole and the organic acid in mortar, a grinder or a mill to obtain a crystal, or adding a solvent to the aripiprazole and the organic acid, followed by grinding and drying to obtain a crystal, wherein the solvent is one or a mixture of two or more selected from a group consisting of aprotic solvents, dimethylformamide, dimethyl sulfoxide, dimethylacetamide, acetonitrile, ether-based solvents, tetrahydrofuran, 1,4-dioxane, alcohol-based solvents, methanol, ethanol, isopropanol, propanol, isobutanol, n-butanol, t-butanol, ethylene glycol, dichloromethane, acetone, methyl ethyl ketone, and distilled water.

In order to achieve the object, a formulation or composition which contains a aripiprazole-organic acid cocrystal prepared according to the above-mentioned method, and which is a pharmaceutically acceptable oral, external, transdermal, transmucosal, injection or inhalation formulation of the cocrystal, and is one or more of tablet, coated tablet, soft capsule, powder, cream, ointment, patch, plaster, injection, aerogel and inhalation formulations.

### Advantageous Effects

According to an aripiprazole-organic acid cocrystal of the present invention, a formulation or composition containing the same, and a preparation method thereof, a novel cocrystal free of hygroscopicity can be obtained by adding an organic acid such as salicylic acid to an anhydrous aripiprazole crystal. In addition, an industrially useful grinding process is provided, which enables the convenience of preparation of the cocrystal and worker's safety to be ensured and allows the preparation of the cocrystal to be performed without needing a special chemical system.

### DESCRIPTION OF DRAWINGS

FIG. 1 shows the molecular structural formula of aripiprazole;

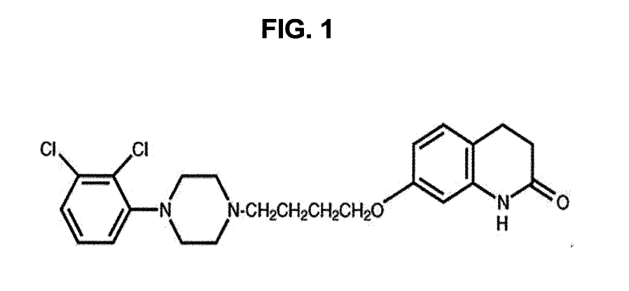

FIG. 2 shows a powder X-ray diffractogram of crystalline aripiprazole that is used as a raw material for preparing an aripiprazole-organic acid cocrystal according to the present invention;

FIG. 3 is a differential scanning calorimetry curve of crystalline aripiprazole that is used as a raw material for preparing an aripiprazole-organic acid cocrystal according to the present invention;

FIG. 4 is the FT-IR spectrum of crystalline aripiprazole that is used as a raw material for preparing an aripiprazole-organic acid cocrystal according to the present invention;

FIG. 5 is a power X-ray diffractogram (simulation) of an aripiprazole-salicylic acid cocrystal according to the present invention;

FIG. 6 shows a power X-ray diffractogram of crystalline salicylic acid that is used as a raw material for preparing an aripiprazole-organic acid cocrystal according to the present invention;

FIG. 7 is a differential scanning calorimetry curve of crystalline aripiprazole that is used as a raw material for preparing an aripiprazole-salicylic acid cocrystal according to the present invention;

FIG. 8 is the FT-IR spectrum of crystalline aripiprazole that is used as a raw material for preparing an aripiprazole-salicylic acid cocrystal according to the present invention;

FIG. 9 shows the molecular structure of an aripiprazole-salicylic acid cocrystal according to the present invention;

FIG. 10 shows the three-dimensional crystalline structure of an aripiprazole-salicylic acid cocrystal according to the present invention;

FIG. 11 shows the intermolecular interaction of an aripiprazole-salicylic acid cocrystal according to the present invention;

FIG. 12 shows the nuclear magnetic resonance spectrum of an aripiprazole-salicylic acid cocrystal according to the present invention;

FIG. 13 shows a powder X-ray diffractogram of an aripiprazole-salicylic acid cocrystal (grinding) according to the present invention;

FIG. 14 is a differential scanning calorimetry curve of an aripiprazole-salicylic acid cocrystal (grinding) according to the present invention;

FIG. 15 shows the FT-IR spectrum of an aripiprazole-salicylic acid cocrystal (grinding) according to the present invention;

FIG. 16 shows a powder X-ray diffractogram of an aripiprazole-adipic acid cocrystal according to the present invention;

FIG. 17 shows a powder X-ray diffractogram of crystalline adipic acid that is used as a raw material for preparing an aripiprazole-adipic acid cocrystal according to the present invention;

FIG. 18 is a differential scanning calorimetry curve of an aripiprazole-adipic acid cocrystal according to the present invention;

FIG. 19 shows the FT-IR spectrum of an aripiprazole-adipic acid cocrystal according to the present invention;

FIG. 20 shows the nuclear magnetic resonance spectrum of an aripiprazole-adipic acid cocrystal according to the present invention;

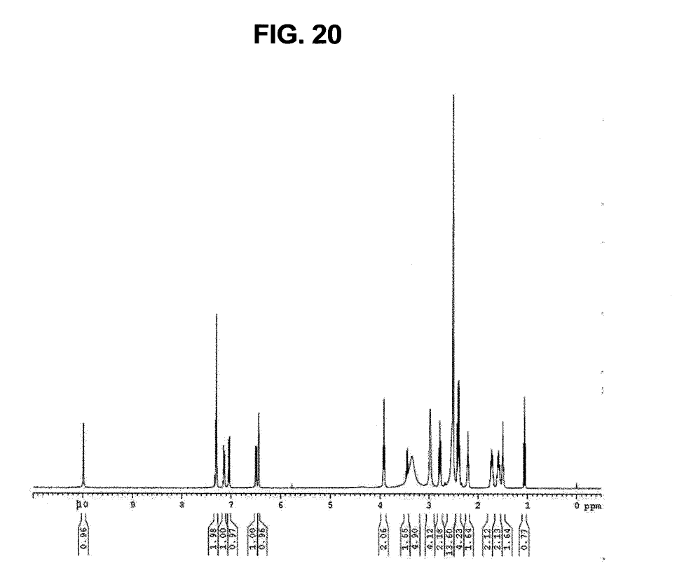

FIG. 21 shows a powder X-ray diffractogram of an aripiprazole-adipic acid cocrystal (grinding) according to the present invention;

FIG. 22 is a differential scanning calorimetry curve of an aripiprazole-adipic acid cocrystal (grinding) according to the present invention;

FIG. 23 shows the FT-IR spectrum of an aripiprazole-adipic acid cocrystal (grinding) according to the present invention;

FIG. 24 shows a powder X-ray diffractogram of an aripiprazole-nicotinic acid cocrystal according to the present invention;

FIG. 25 shows a powder X-ray diffractogram of crystalline nicotinic acid that is used as a raw material for preparing an aripiprazole-nicotinic acid cocrystal according to the present invention;

FIG. 26 shows a differential scanning calorimetry curve of an aripiprazole-nicotinic acid cocrystal according to the present invention;

FIG. 27 shows the FT-IR spectrum a differential scanning calorimetry curve of an aripiprazole-nicotinic acid cocrystal according to the present invention;

FIG. 28 shows the nuclear magnetic resonance of an aripiprazole-nicotinic acid cocrystal according to the present invention;

FIG. 29 shows a powder X-ray diffraction spectrum of an aripiprazole-nicotinic acid cocrystal (grinding) according to the present invention;

FIG. 30 shows a differential scanning calorimetry curve of an aripiprazole-nicotinic acid cocrystal (grinding) according to the present invention;

FIG. 31 shows the FT-IR spectrum of an aripiprazole-nicotinic acid cocrystal (grinding) according to the present invention;

FIG. 32 shows a powder X-ray diffractogram of an aripiprazole-benzenesulfonic acid cocrystal according to the present invention;

FIG. 33 is a differential scanning calorimetry curve of an aripiprazole-benzenesulfonic acid cocrystal according to the present invention;

FIG. 34 shows the FT-IR spectrum of an aripiprazole-benzenesulfonic acid cocrystal according to the present invention;

FIG. 35 shows the nuclear magnetic resonance spectrum of an aripiprazole-benzenesulfonic acid cocrystal according to the present invention;

FIG. 36 shows a powder X-ray diffractogram of an aripiprazole-terephthalic acid cocrystal according to the present invention;

FIG. 37 shows a powder X-ray diffractogram of crystalline terephthalic acid that is used as a raw material for preparing an aripiprazole-terephthalic acid cocrystal according to the present invention;

FIG. 38 is a differential scanning calorimetry curve of an aripiprazole-terephthalic acid cocrystal according to the present invention;

FIG. 39 shows the FT-IR spectrum of an aripiprazole-terephthalic acid cocrystal according to the present invention;

FIG. 40 shows the nuclear magnetic resonance spectrum of an aripiprazole-terephthalic acid cocrystal according to the present invention;

FIG. 41 shows a powder X-ray diffractogram of an aripiprazole-terephthalic acid cocrystal (grinding) according to the present invention;

FIG. 42 is a differential scanning calorimetry curve of an aripiprazole-terephthalic acid cocrystal (grinding) according to the present invention; and

FIG. 43 shows the FT-IR spectrum of an aripiprazole-terephthalic add cocrystal (grinding) according to the present invention.

### BEST MODE

The present invention may be subjected to various modifications, and may have various embodiments. Specific embodiments are illustrated in drawings, and will be described in the detailed description of the present invention. However, this is not intended to limit the present invention to specific embodiments. It should be understood that the present invention includes all modifications, equivalents or replacements that fall within the spirit and technical range of the present invention, and the scope of the present invention is not limited to the following embodiments.

An aripiprazole-organic acid cocrystal according to the present invention consists of aripiprazole and an organic acid, wherein the organic acid is one or more of salicylic acid, adipic acid, benzenesulfonic acid, nicotinic acid, and terephthalic acid, and the molecular ratio of aripiprazole to the organic acid is 1:1 or 2:1.

A method for preparing an aripiprazole-organic acid cocrystal according to the present invention comprises forming a cocrystal from aripiprazole and an organic acid by a solvent process or a grinding process, wherein the organic acid is one or more of salicylic acid, adipic acid, benzenesulfonic acid, nicotinic acid, terephthalic acid and the molecular ratio of aripiprazole to the organic acid in the cocrystal is 1:1 or 2:1.

In an embodiment, the method for preparing the aripiprazole-organic acid cocrystal according to the present invention comprises the steps of: dissolving aripiprazole and the organic acid in a solvent to form a solution; and either crystallizing the solution under cold conditions at a temperature of 0˜10° C., or evaporating the solvent from the solution, thereby obtaining the cocrystal, wherein the solvent is one or a mixture of two or more selected from among aprotic solvents, dimethylformamide, dimethyl sulfoxide, dimethylacetamide, acetonitrile, ether-based solvents, tetrahydrofuran, 1,4-dioxane, alcohol-based solvents, methanol, ethanol, isopropanol, propanol, isobutanol, n-butanol, t-butanol, ethylene glycol, dichloromethane, acetone, methyl ethyl ketone, and distilled water.

In another embodiment, the method for preparing the aripiprazole-organic acid cocrystal according to the present invention comprises grinding aripiprazole and the organic acid in mortar, a grinder or a mill to obtain a crystal, or adding a solvent to aripiprazole and the organic acid, followed by grinding and drying to obtain a crystal, wherein the solvent is one or a mixture of two or more selected from among aprotic solvents, dimethylformamide, dimethyl sulfoxide, dimethylacetamide, acetonitrile, ether-based solvents, tetrahydrofuran, 1,4-dioxane, alcohol-based solvents, methanol, ethanol, isopropanol, propanol, isobutanol, n-butanol, t-butanol, ethylene glycol, dichloromethane, acetone, methyl ethyl ketone, and distilled water.

A formulation or composition containing the aripiprazole-organic acid cocrystal according to the present invention is a pharmaceutically acceptable oral, transdermal, transmucosal, injection or inhalation formulation of the cocrystal prepared by the above method, and is one or more of tablet, coated tablet, soft capsule, powder, cream, ointment, patch, plaster, injection, aerogel and inhalation formulations.

As described above, the present invention provides an aripiprazole-organic acid cocrystal, a preparation method thereof, and a formulation or composition containing the aripiprazole-organic acid cocrystal together with an excipient. The method for preparing the aripiprazole-organic acid cocrystal according to the present invention is performed without an adsorbent such as a polymer, and thus enables a stable aripiprazole-organic acid cocrystal to be easily obtained, unlike the case in which a drug is released from an adsorbent together with a solid dispersion so that it is partially recrystallized to reduce its solubility.

An important factor in the preparation of the aripiprazole-organic acid cocrystal is the control of hydrogen bonding. Hydrogen bonding is the most important motive for formation of the cocrystal. In the absence of an ionic bond such as salt, a molecule interacts competitively with a hydrogen bond partner such as a solvent. In addition, because a hydrogen bond between the same molecules also exists, a mixture of different crystals or a solvate is easily produced when hydrogen bonding is not suitably controlled. In the present invention, it was attempted to prepare a cocrystal through typical hydrogen binding between the amide functional group of aripiprazole and the —N—H . . . O═C—(I) and —C═O . . . H—O—C—(II) of the carboxyl group of salicylic acid. The method of preparing the aripiprazole-organic acid cocrystal by the solvent process is as follows. (A) Aripiprazole and an organic acid are dissolved in a suitable solvent to make a solution. (B) An anti-solvent is added, or the solvent is slowly evaporated in a hood. (C) The temperature of the solution is reduced to form a cocrystal consisting of aripiprazole and the organic acid, or the solution is allowed to stand at room temperature until a crystal is formed by evaporation of the solvent. (D) The solvent is removed by filtration, and a cocrystal is collected. (E) The cocrystal is confirmed by instrumental analysis. Meanwhile, the method of preparing the aripiprazole-organic acid cocrystal by the grinding process is as follows. (A) Aripiprazole and an organic acid are placed in mortar or a grinder at an equivalent ratio. (B) The placed aripiprazole and organic acid are ground using a suitable tool. If necessary, a slight amount of a solvent is added before grinding. (C) The cocrystal is confirmed by instrumental analysis. After instrumental analysis, additional grinding may be performed, if necessary.

The cocrystal can be analyzed by powder X-ray diffraction (PXRD), differential scanning calorimetry (DSC), FT-IR spectroscopy, nuclear magnetic resonance (NMR) spectroscopy and the like. A more reliable analysis method is a direct measurement method. In the present invention, a single crystal of an aripiprazole-organic acid cocrystal (1:1) was prepared and analyzed by powder X-ray diffraction (PXRD) to confirm the three-dimensional crystalline structure of the cocrystal, the conformation of each molecule, and the hydrogen bonding pattern regarding the fundamental interaction between the molecules.

Each crystallographic axis and the angle between axes in the monoclinic form [Space Group P2(1)/c] are as follows (the parenthesized numerals indicate error):

a=15.0588(8) Å

b=9.6145(5) Å

c=21.0025(12) Å

alpha=gamma=90°

beta=106.737(1)°

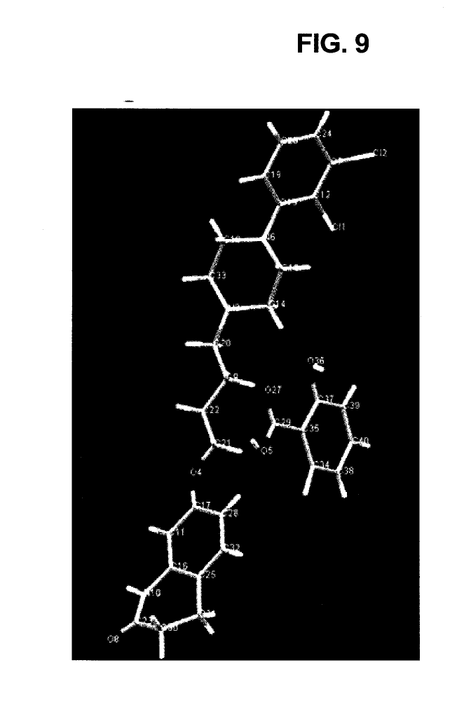

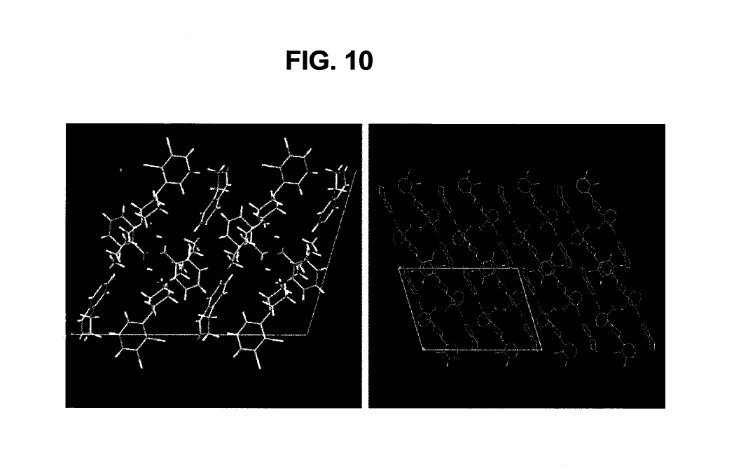

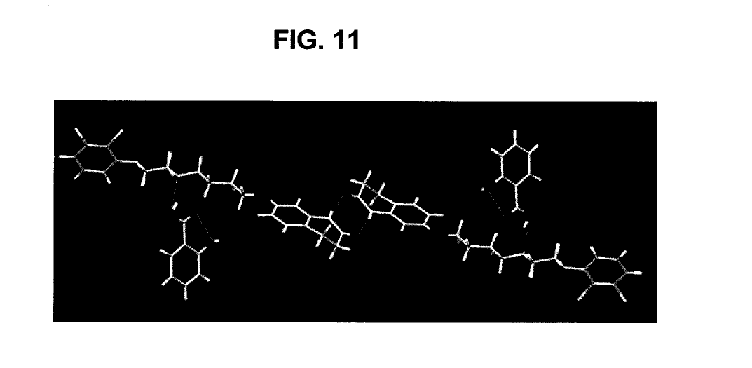

The molecular structure and cocrystal structure obtained by analysis, and the hydrogen bonding pattern, are shown in FIGS. 9 to 11.

In the present invention, PXRD data were acquired using CuKα-1 X-rays (wavelength: 1.541 Å) generated by a Bruker Axe D8 Advance powder X-ray diffractometer. DSC data were obtained in a temperature range of 50˜200° C. at a heating rate of 10° C./min using DSC Q100 (TA Instruments, Inc.). FT-IR data were obtained for a sample pellet by the KBr method at a resolution of 4 cm⁻¹ in the range of 450˜4,000 cm⁻¹ using a Perkin Elmer System Spectrum 1 spectrophotometer. ¹H-NMR data were obtained using JNM-AL300 (JEOL Ltd.) at 400 MHz on a sample dissolved in dimethyl sulfoxide (DMSO-d6). The structural analysis of a single crystal was performed by acquiring and analyzing data using the SMART APEX II CCD X-ray diffractometer to determine the three-dimensional structure.

### Example 1

### Preparation of Aripiprazole-Salicylic Acid Cocrystal by Solvent Process

500 mg of aripiprazole was mixed with 154 mg of salicylic acid, and then 10 mL of ethanol, 2 mL of dimethyl sulfoxide, 10 mL of dichloromethane, and 1 mL of distilled water were added thereto and sufficiently mixed by vortex. The mixture was dissolved in a sonicator. Then, it was cold-stored at 4° C., and the precipitated solid was collected and dried at room temperature.

### Analysis Example 1

### Crystalline Aripiprazole and Salicylic Acid and the Prepared Aripiprazole-Salicylic Acid Cocrystal were Comparatively Analyzed

<Powder X-Ray Diffraction Analysis>

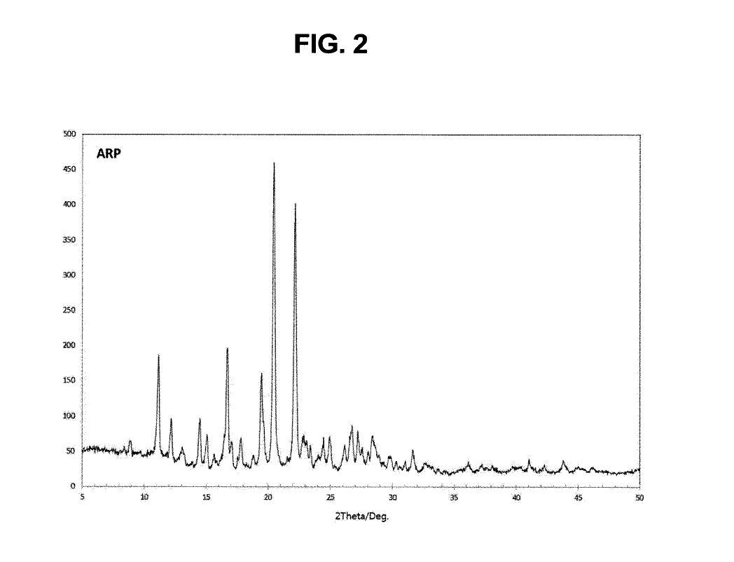

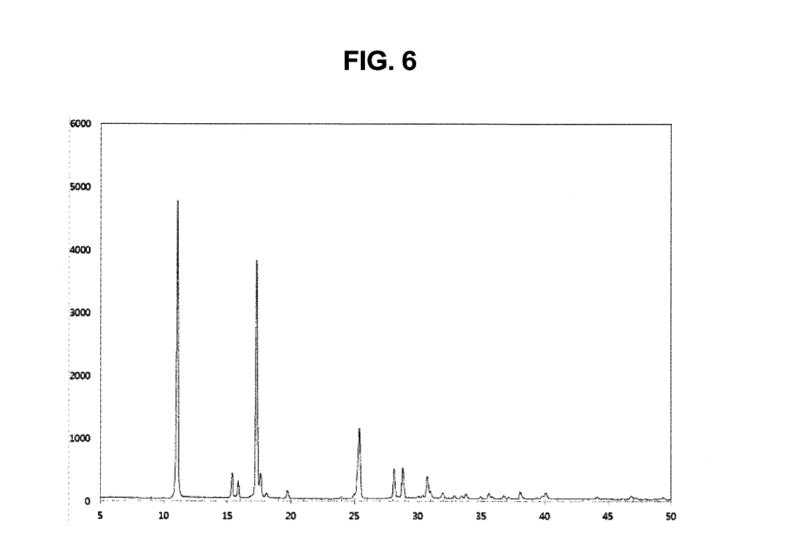

From a comparison between FIGS. 2, 5 and 6, it can be seen that the three figures show distinct crystalline forms, indicating that crystalline aripiprazole and salicylic acid and the prepared aripiprazole-salicylic acid cocrystal are different crystals.

<FT-IR Spectroscopy>

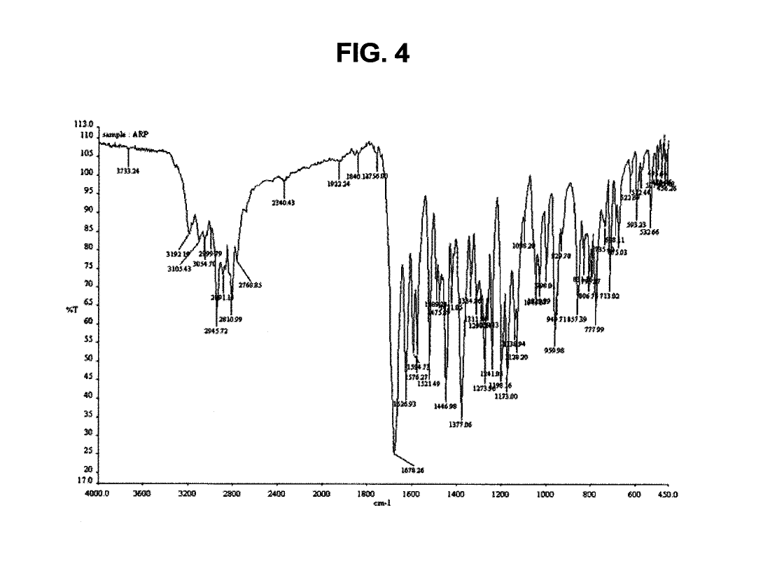

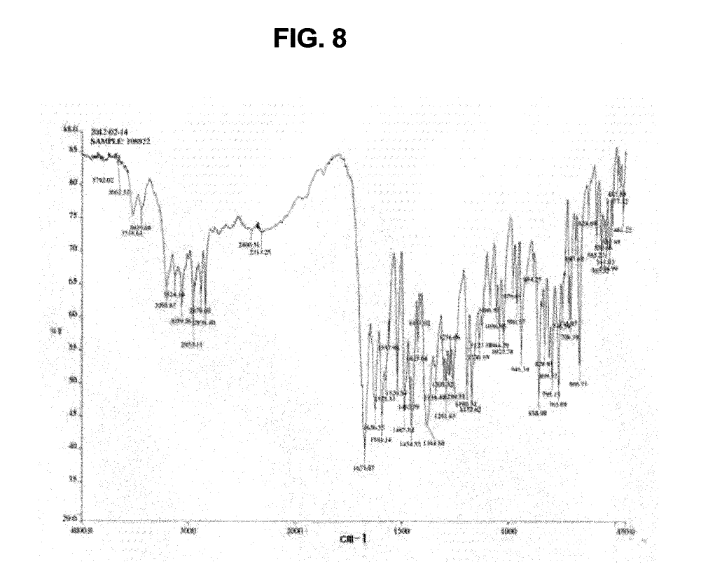

As can be seen in FIGS. 4 and 8, the characteristic bands of the aripiprazole-salicylic acid cocrystal appear at wavenumbers of 666, 764, 858, 941, 1173, 1194, 1384, 1454, 1626, 1674, 2953, 3059, 3202, 3440, and 3519±5 cm⁻¹.

<Differential Scanning Calorimetry (DSC)>

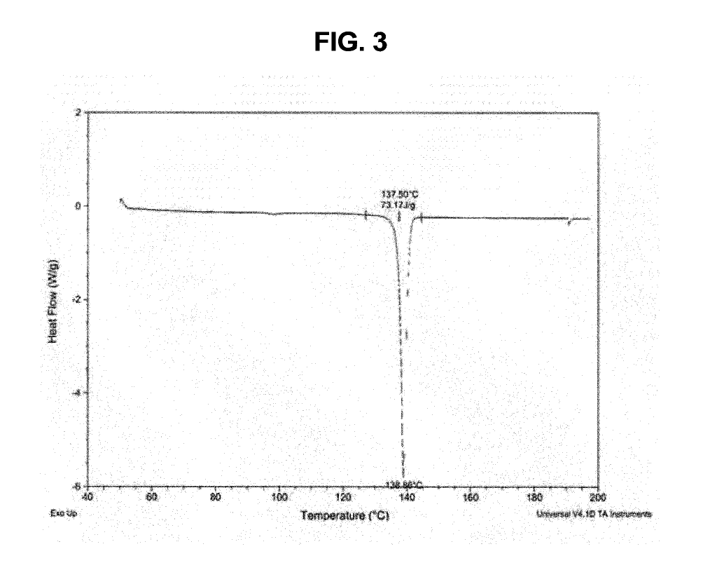

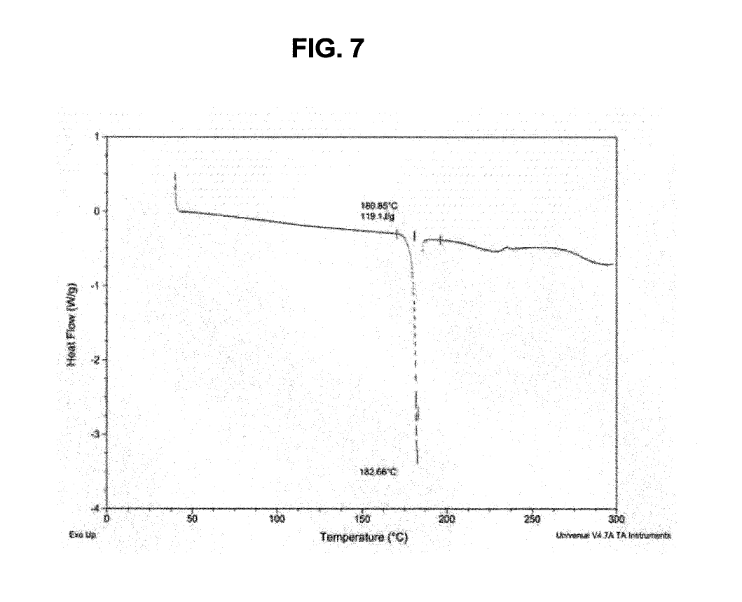

As can be seen in FIGS. 3 and 7, crystalline aripiprazole shows an endothermic peak at about 137.5° C. (FIG. 3), suggesting that it is crystalline. Also, as shown in FIG. 7, the aripiprazole-salicylic acid cocrystal shows a new endothermic peak at about 180.8° C., suggesting that a new crystal was produced.

<Nuclear Magnetic Resonance Spectrum>

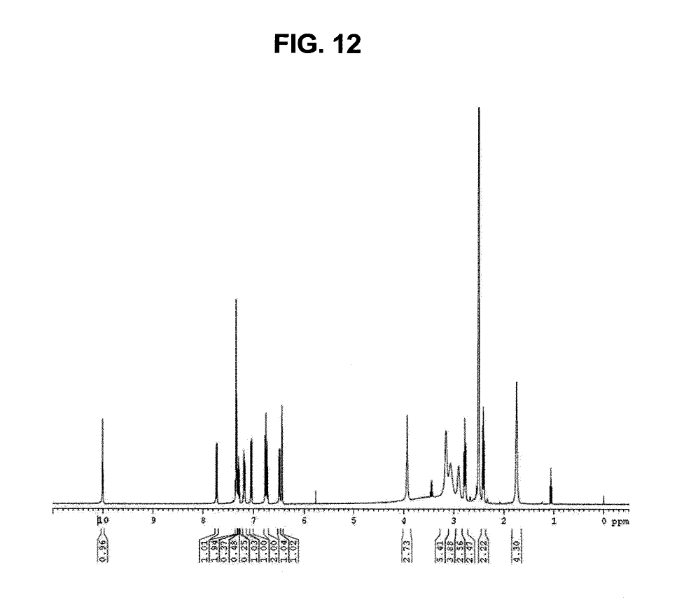

The peaks in FIG. 12 indicate that aripiprazole coexists with salicylic acid, and that the ratio of hydrogen between the molecules is 1:1.

<Single Crystal X-Ray Diffraction Analysis of Single Crystal>

To confirm the prepared aripiprazole-salicylic acid cocrystal, single crystal X-ray diffraction analysis was conducted.

As can be seen in FIGS. 9 to 11, a 1:1 cocrystal is formed by hydrogen bonding between the two molecules, and the conformation of each molecule is also shown.

### Example 2

### Preparation of Aripiprazole-Salicylic Acid Cocrystal by Grinding Process

500 mg of aripiprazole was mixed with 154 mg of salicylic acid, and the mixture was placed in agate mortar, and ground uniformly with an agate pestle for 10 minutes. Alternatively, 0.2 mL of methanol or DMSO was added to the mixture, which was then ground for 10 minutes and dried overnight in a hood.

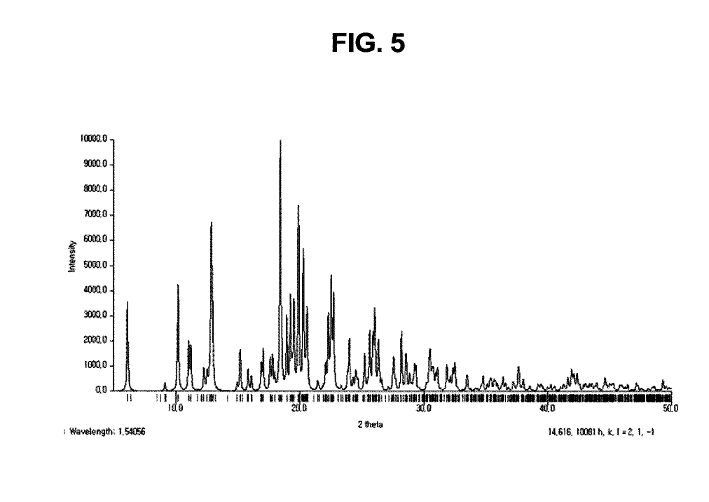

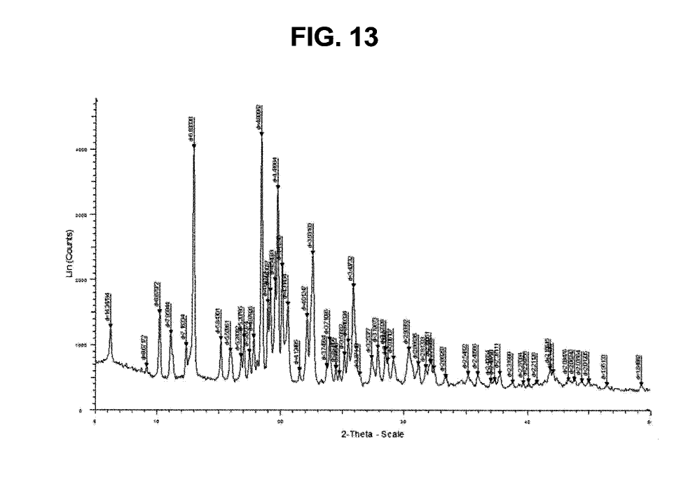

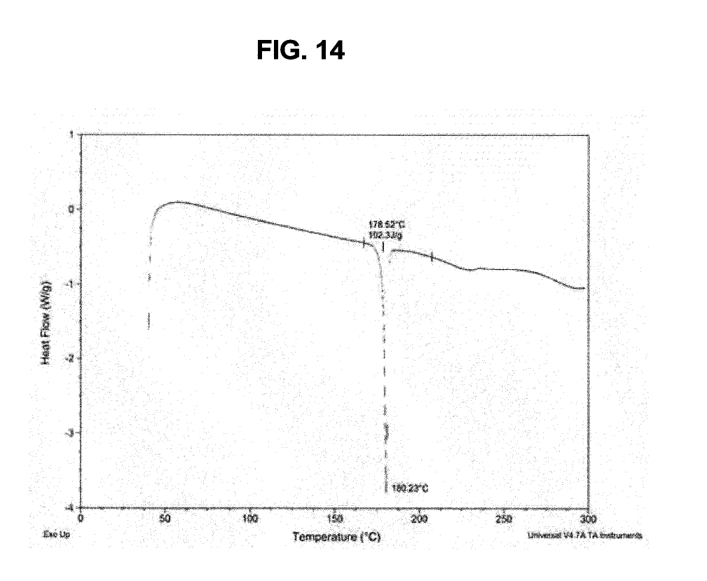

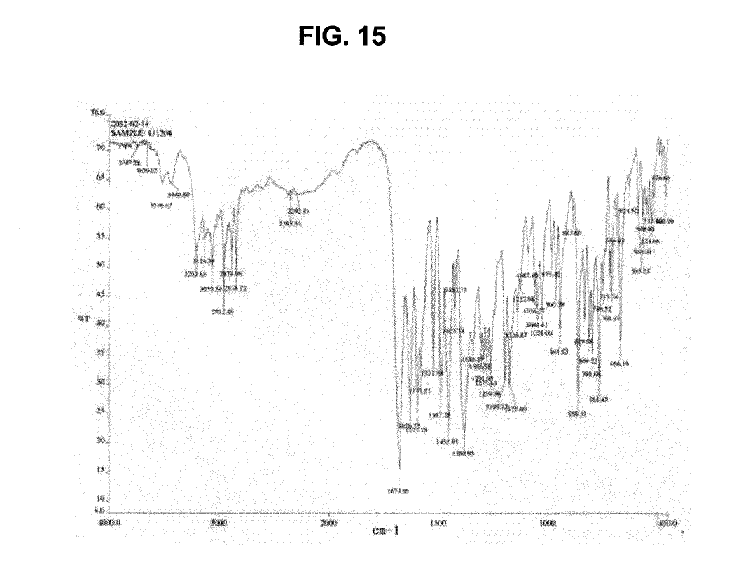

From a comparison between FIGS. 13 to 15 with FIGS. 5, 7 and 8, respectively, it can be seen that the cocrystal prepared in this Example is the same as the cocrystal prepared in Example 1.

### Example 3

### Preparation of Aripiprazole-Adipic Acid Cocrystal by Solvent Process

500 mg of aripiprazole was mixed with 81.5 mg of adipic acid, and then 5 mL of ethanol and 5 mL of dichloromethane were added thereto and sufficiently mixed by vortex. The mixture was dissolved in a sonicator. Then, the solvent was evaporated slowly in a hood, and the precipitated solid was collected and dried at room temperature.

### Analysis Example 2

### Crystalline Aripiprazole and Adipic Acid and the Prepared Aripiprazole-Adipic Acid Cocrystal were Comparatively Analyzed

<Powder X-Ray Diffraction Analysis>

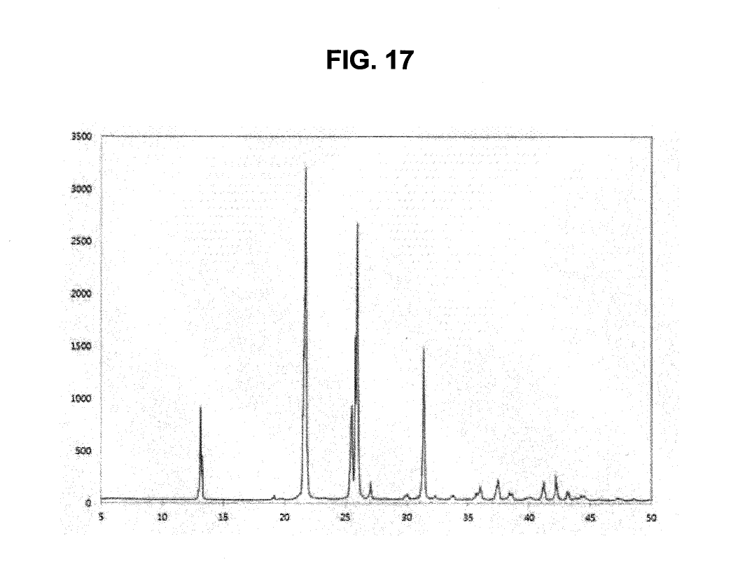

From a comparison between FIGS. 2, 16 and 17, it can be seen that the three figures show distinct crystalline forms, indicating that crystalline aripiprazole and adipic acid and the prepared aripiprazole-adipic acid cocrystal are different crystals.

The characteristic peaks of the aripiprazole-adipic acid cocrystal mainly appear at 2 theta values of 9.7, 14.5, 17.5, 18.3, 18.9, 19.8, 22.8, 24.2 and 24.6±0.2°.

<FT-IR Spectroscopy>

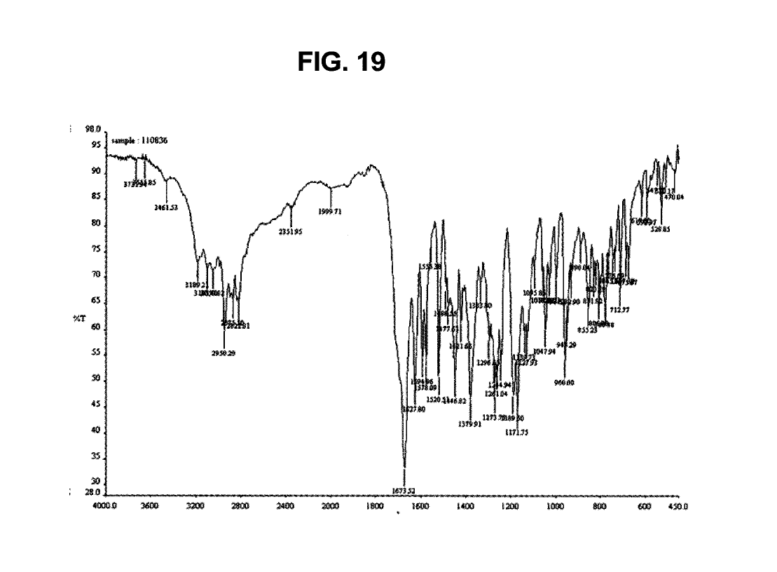

As can be seen in FIGS. 4 and 19, the characteristic bands of the aripiprazole-adipic acid cocrystal appear at wavenumbers of 667, 779, 855, 960, 1172, 1190, 1380, 1447, 1628, 1673, 2950, 3047, 3189 and 3462±5 cm⁻¹.

<Differential Scanning Calorimetry (DSC)>

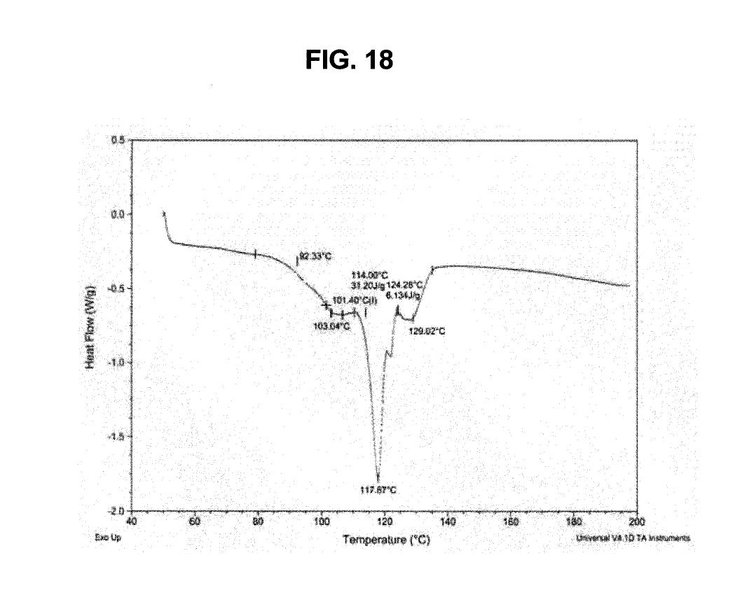

As can be seen in FIGS. 3 and 18, crystalline aripiprazole shows an endothermic peak at about 137.5° C. (FIG. 3), suggesting that it is crystalline. Also, as shown in FIG. 18, the aripiprazole-adipic acid cocrystal shows a new endothermic peak at about 114.0° C., suggesting that a new crystal was produced.

<Nuclear Magnetic Resonance Spectrum>

The peaks in FIG. 12 indicate that aripiprazole coexists with adipic acid, and that the ratio of hydrogen between the molecules is 2:1.

### Example 4

### Preparation of Aripiprazole-Adipic Acid Cocrystal by Grinding Process

500 mg of aripiprazole was mixed with 81.5 mg of adipic acid, and the mixture was placed in agate mortar, and ground uniformly with an agate pestle for 10 minutes. Alternatively, 0.2 mL of methanol was added to the mixture, which was then ground for 10 minutes and dried overnight in a hood.

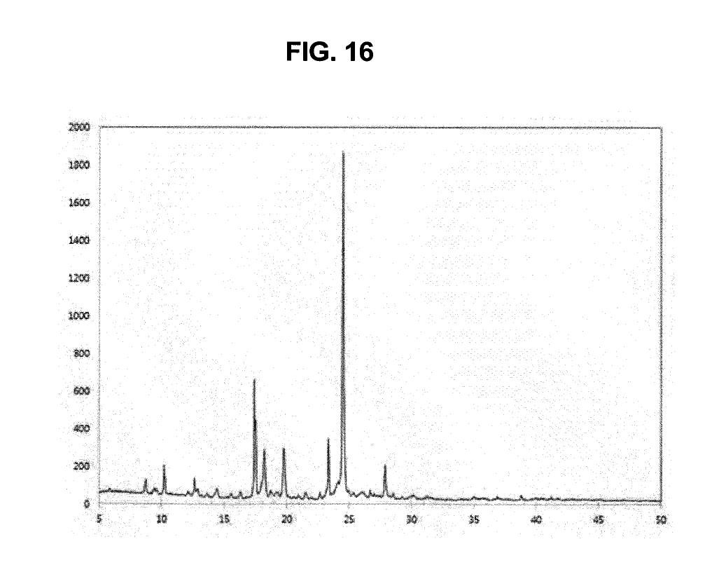

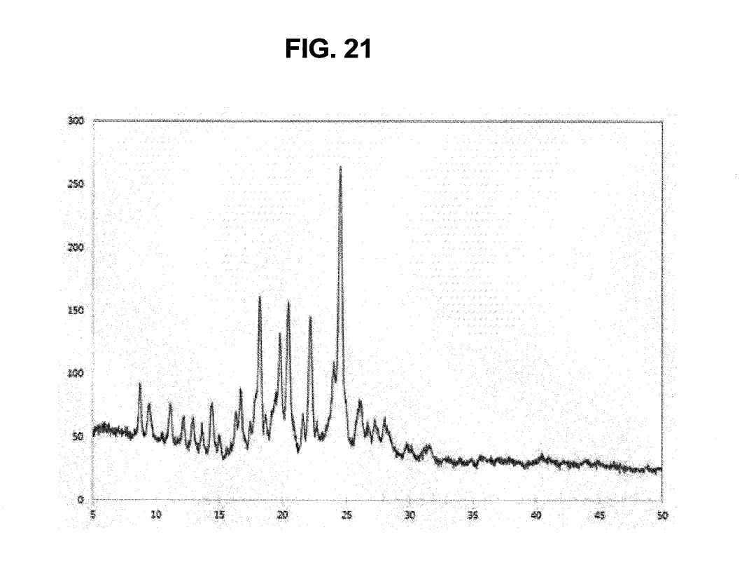

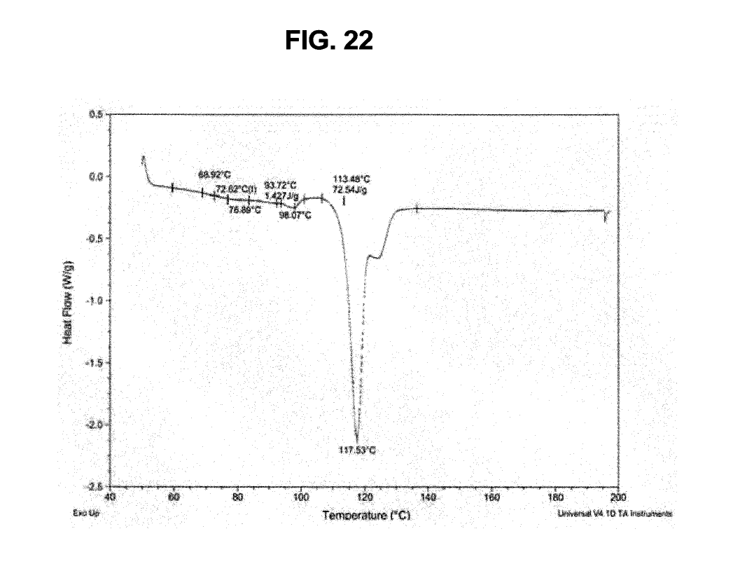

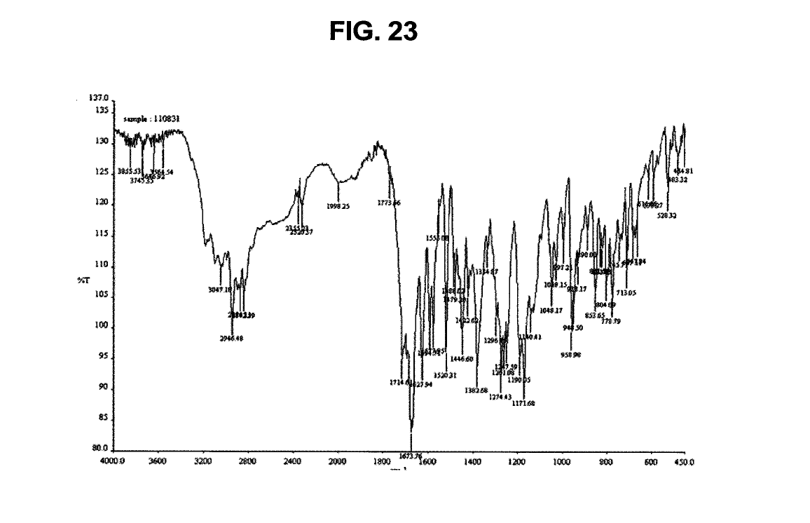

From a comparison between FIGS. 21 to 23 with FIGS. 16, 18 and 19, respectively, it can be seen that the cocrystal prepared in this Example is the same as the cocrystal prepared in Example 3.

### Example 5

### Preparation of Aripiprazole-Nicotinic Acid Cocrystal by Solvent Process

500 mg of aripiprazole was mixed with 137.3 mg of nicotinic acid, and then 5 mL of ethanol and 5 mL of dichloromethane were added thereto and sufficiently mixed by vortex. The mixture was dissolved in a sonicator. Then, the solvent was evaporated slowly in a hood, and the precipitated solid was collected and dried at room temperature.

### Analysis Example 3

### Crystalline Aripiprazole and Nicotinic Acid and the Prepared Aripiprazole-Nicotinic Acid Cocrystal were Comparatively Analyzed

<Powder X-Ray Diffraction Analysis>

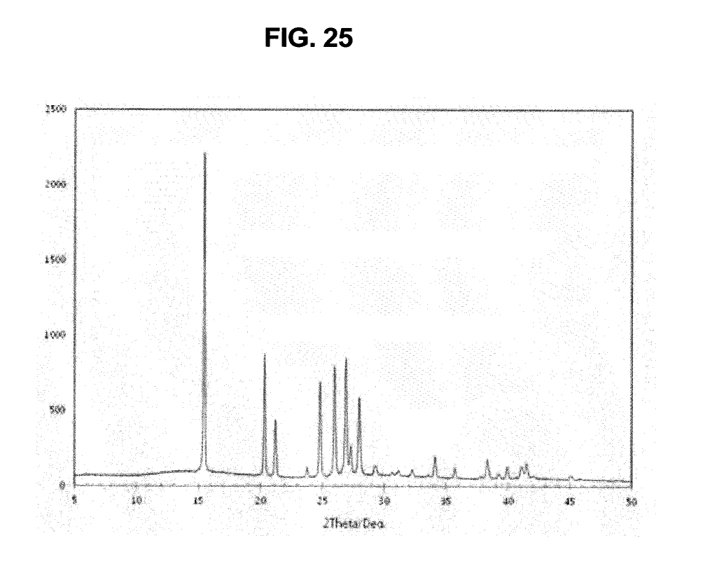

From a comparison between FIGS. 2, 24 and 25, it can be seen that the three figures show distinct crystalline forms, indicating that crystalline aripiprazole and nicotinic acid and the prepared aripiprazole-nicotinic acid cocrystal are different crystals. The characteristic peaks of the aripiprazole-nicotinic acid cocrystal mainly appear at 2 theta values of 12.9, 15.6, 17.6, 18.3, 19.6, 20.5, 22.7, 25.0, 27.0 and 28.0±0.2°.

<FT-IR Spectroscopy>

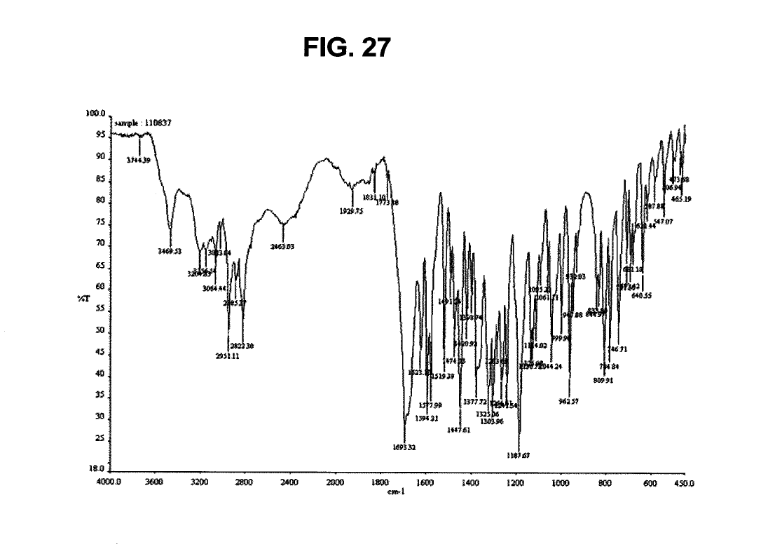

As can be seen in FIGS. 4 and 27, the characteristic bands of the aripiprazole-nicotinic acid cocrystal appear at wavenumbers of 641, 784, 844, 962, 1187, 1241, 1377, 1448, 1623, 1693, 2463, 2822, 2951, 3064, 3204 and 3469±5 cm⁻¹.

<Differential Scanning Calorimetry (DSC)>

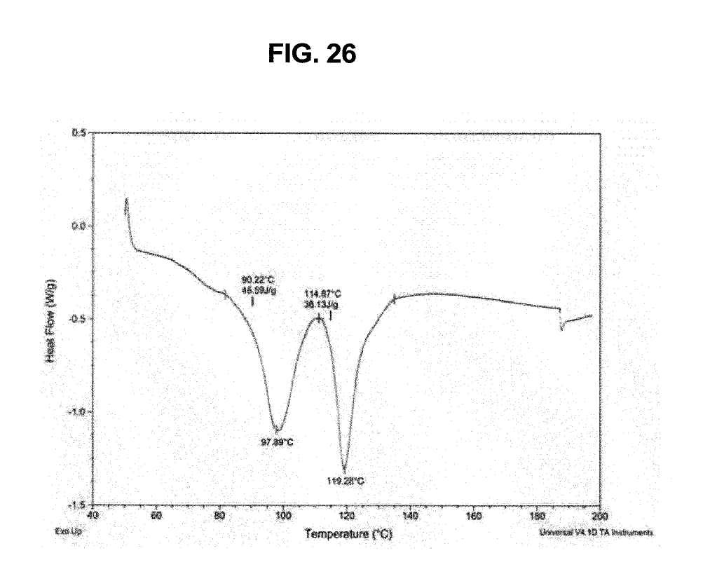

As can be seen in FIGS. 3 and 26, crystalline aripiprazole shows an endothermic peak at about 137.5° C. (FIG. 3), suggesting that it is crystalline. Also, as shown in FIG. 26, the aripiprazole-nicotinic acid cocrystal shows new endothermic peaks at about 90° C. and 114.0° C., suggesting that a new crystal was produced.

<Nuclear Magnetic Resonance Spectrum>

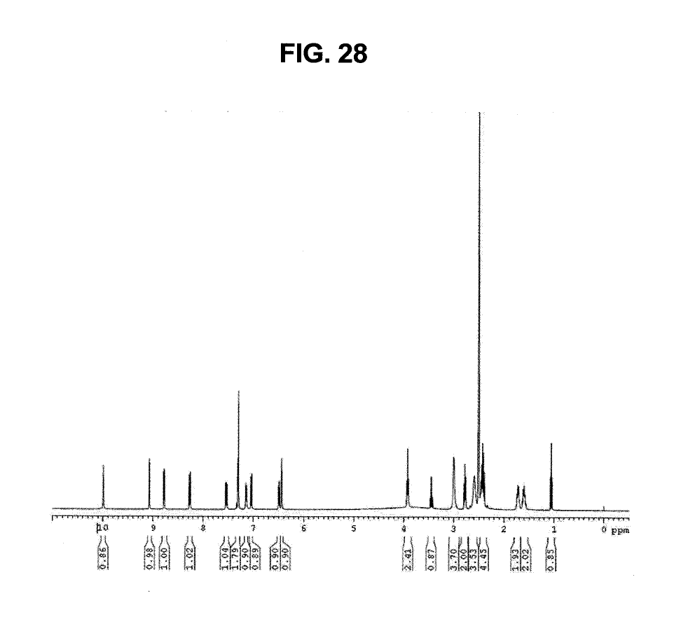

The peaks in FIG. 28 indicate that aripiprazole coexists with nicotinic acid, and that the ratio of hydrogen between the molecules is 1:1.

### Example 6

### Preparation of Aripiprazole-Nicotinic Acid Cocrystal by Grinding Process

500 mg of aripiprazole was mixed with 137.3 mg of nicotinic acid, and the mixture was placed in agate mortar, and ground uniformly with an agate pestle for 10 minutes. Alternatively, 0.2 mL of methanol was added to the mixture, which was then ground for 10 minutes and dried overnight in a hood.

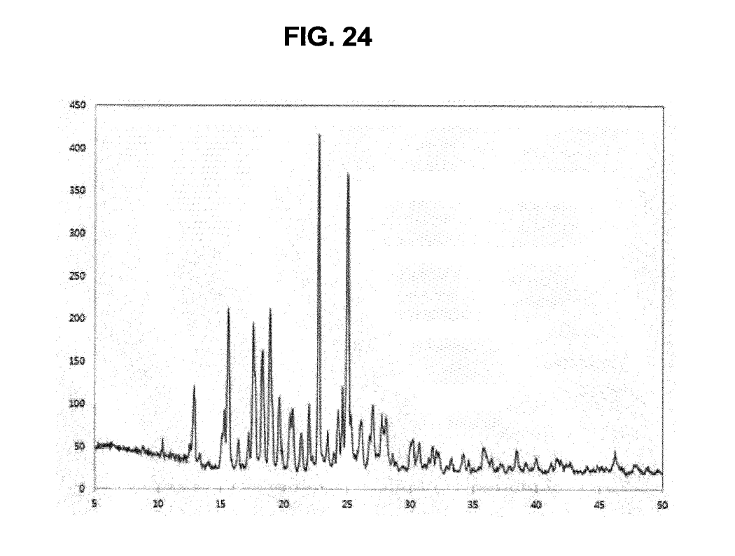

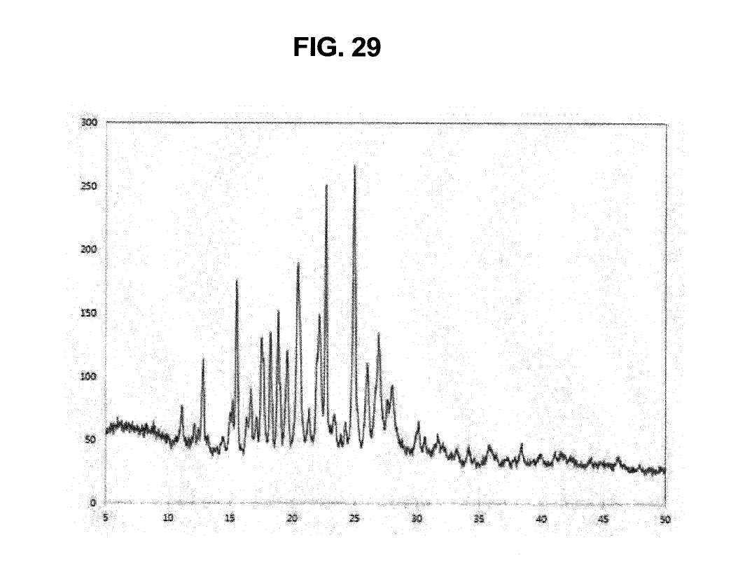

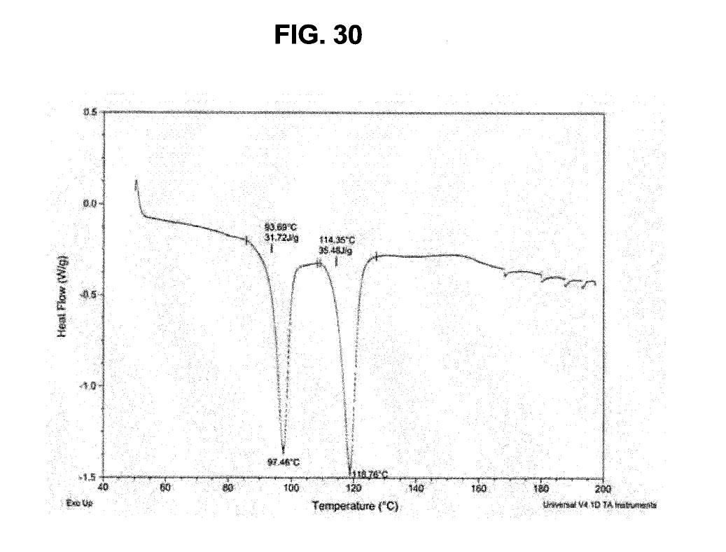

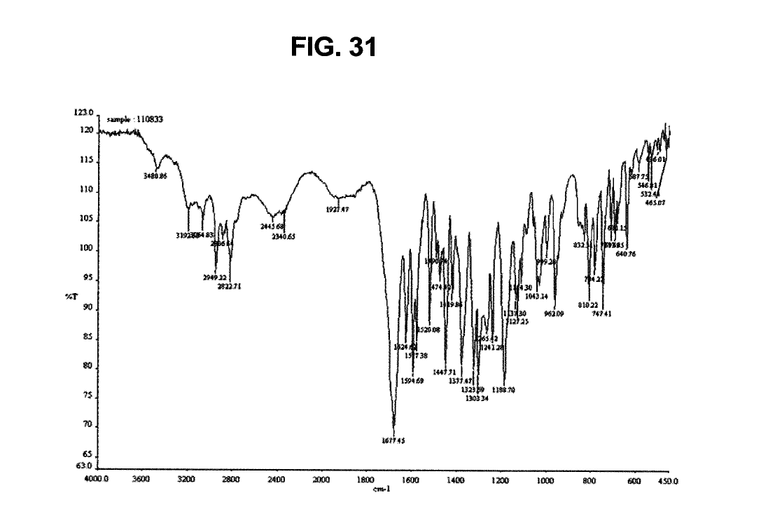

From a comparison between FIGS. 29 to 31 with FIGS. 24, 26 and 27, respectively, it can be seen that the cocrystal prepared in this Example is the same as the cocrystal prepared in Example 5.

### Example 7

### Preparation of Aripiprazole-Benzenesulfonic Acid Cocrystal by Solvent Process

500 mg of aripiprazole was mixed with 176.4 mg of benzenesulfonic acid, and then 5 mL of dichloromethane was added thereto and sufficiently mixed by vortex. The mixture was dissolved in a sonicator. Then, 5 mL of ethanol was added thereto, stored in a refrigerator at 4° C., and the precipitated solid was collected and dried at room temperature.

### Analysis Example 4

### Crystalline Aripiprazole and Benzenesulfonic Acid and the Prepared Aripiprazole-Benzenesulfonic Acid Cocrystal were Comparatively Analyzed

<Powder X-Ray Diffraction Analysis>

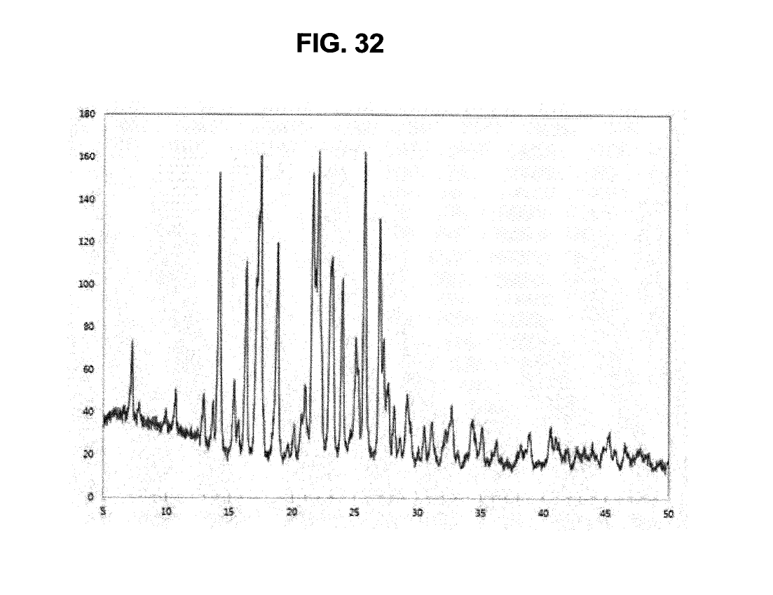

From a comparison between FIGS. 2 and 32, it can be seen that the two figures show distinct crystalline forms, indicating that crystalline aripiprazole and benzenesulfonic acid and the prepared aripiprazole-benzenesulfonic acid cocrystal are different crystals. The benzenesulfonic acid was an amorphous semi-solid in the initial stage. The characteristic peaks of the aripiprazole-benzenesulfonic acid cocrystal mainly appear at 2 theta values of 7.2, 14.2, 16.3, 17.4, 18.8, 21.7, 22.1, 23.2, 24.0, 25.8 and 27.0±0.2°.

<FT-IR Spectroscopy>

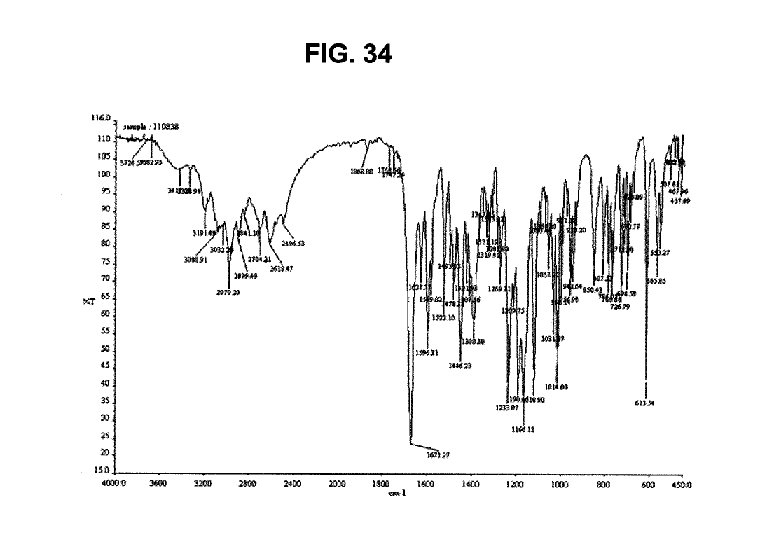

As can be seen in FIGS. 4 and 34, the characteristic bands of the aripiprazole-benzenesulfonic acid cocrystal appear at wavenumbers of 614, 784, 850, 957, 1166, 1233, 1388, 1446, 1627, 1671, 2496, 2618, 2979, 3080, 3191 and 3413±5 cm⁻¹.

<Differential Scanning Calorimetry (DSC)>

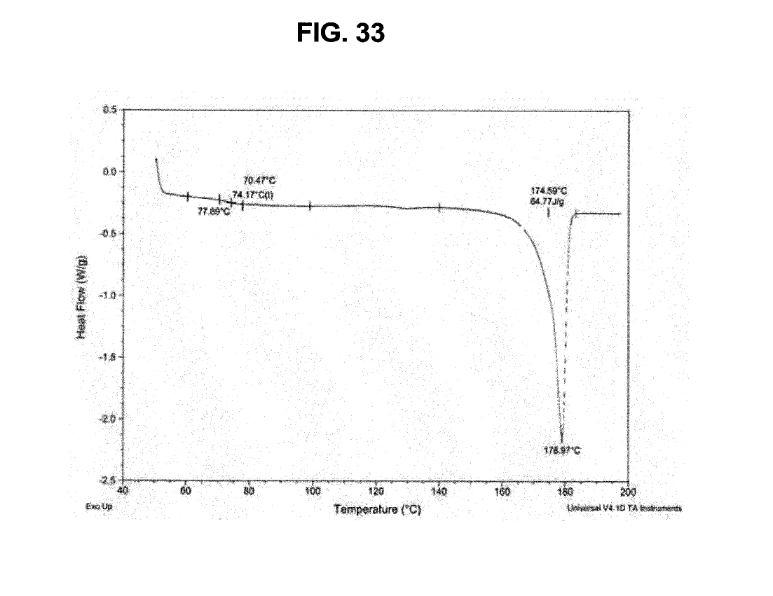

As can be seen in FIGS. 3 and 33, crystalline aripiprazole shows an endothermic peak at about 137.5° C. (FIG. 3), suggesting that it is crystalline. Also, as shown in FIG. 33, the aripiprazole-benzenesulfonic acid cocrystal shows a new endothermic peak at about 174.6° C., suggesting that a new crystal was produced.

<Nuclear Magnetic Resonance Spectrum>

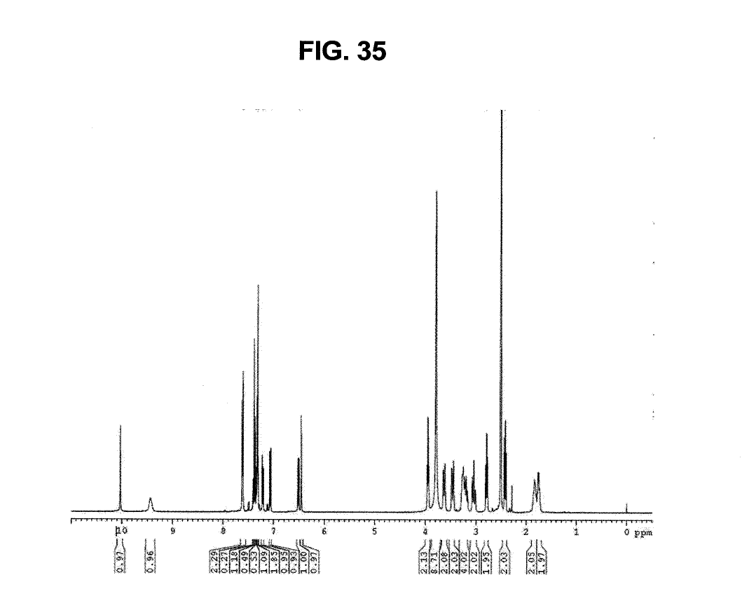

The peaks in FIG. 35 indicate that aripiprazole coexists with benzenesulfonic acid, and that the ratio of hydrogen between the molecules is 1:1.

### Example 8

### Preparation of Aripiprazole-Terephthalic Acid Cocrystal by Solvent Process

500 mg of aripiprazole was mixed with 92.6 mg of terephthalic acid, and then 10 mL of ethanol, 2 mL of dimethyl sulfoxide, 10 mL of dichloromethane and 1 mL of distilled water were added thereto and sufficiently mixed by vortex. The mixture was dissolved in a sonicator. Then, the solvent was evaporated slowly in a hood, and the precipitated solid was collected and dried at room temperature.

### Analysis Example 5

### Crystalline Aripiprazole and Terephthalic Acid and the Prepared Aripiprazole-Terephthalic Acid Cocrystal were Comparatively Analyzed

<Powder X-Ray Diffraction Analysis>

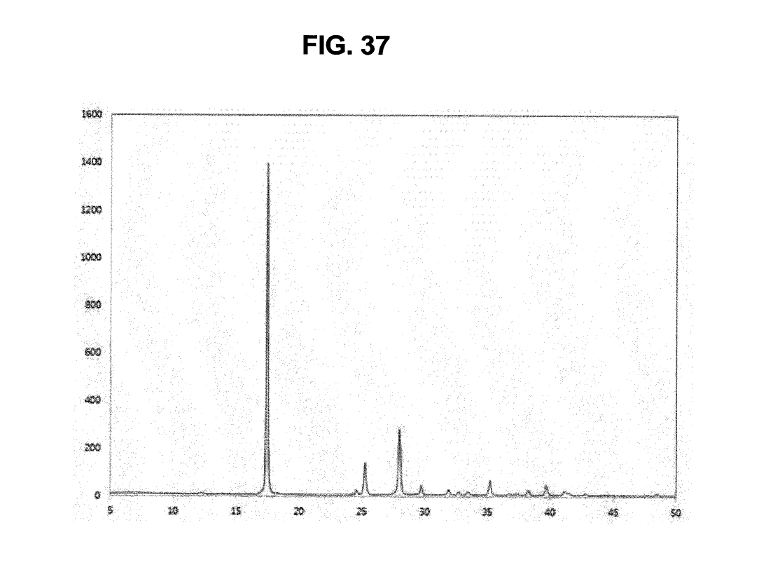

From a comparison between FIGS. 2, 36 and 37, it can be seen that the three figures show distinct crystalline forms, indicating that crystalline aripiprazole and terephthalic acid and the prepared aripiprazole-terephthalic acid cocrystal are different crystals. The characteristic peaks of the aripiprazole-terephthalic acid cocrystal mainly appear at 2 theta values of 12.8, 16.7, 17.1, 17.5, 18.2, 19.5, 20.5, 22.1, 22.6, 24.9, 27.6 and 28.0±0.2°.

<FT-IR Spectroscopy>

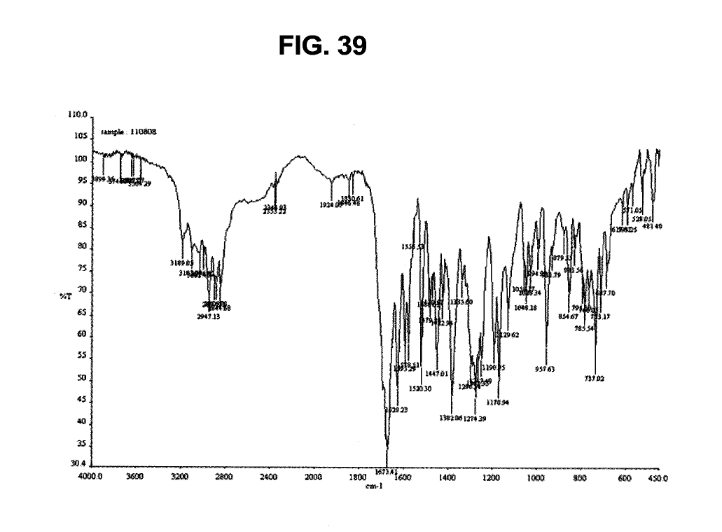

As can be seen in FIGS. 4 and 39, the characteristic bands of the aripiprazole-terephthalic acid cocrystal appear at wavenumbers of 688, 737, 854, 957, 1171, 1274, 1382, 1447, 1628, 1673, 2844, 2947, 3063 and 3189±5 cm⁻¹.

<Differential Scanning Calorimetry (DSC)>

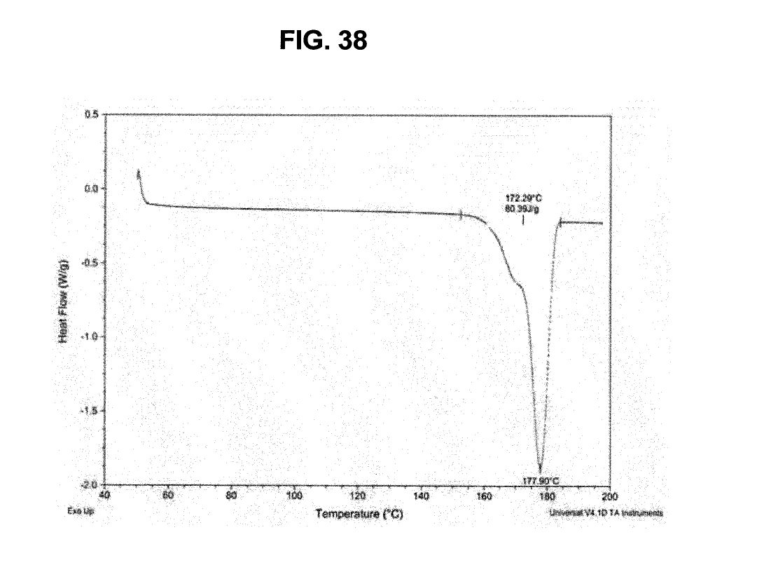

As can be seen in FIGS. 3 and 38, crystalline aripiprazole shows an endothermic peak at about 137.5° C. (FIG. 3), suggesting that it is crystalline. Also, as shown in FIG. 38, the aripiprazole-terephthalic acid cocrystal shows a new endothermic peak at about 174.60° C., suggesting that a new crystal was produced.

<Nuclear Magnetic Resonance Spectrum>

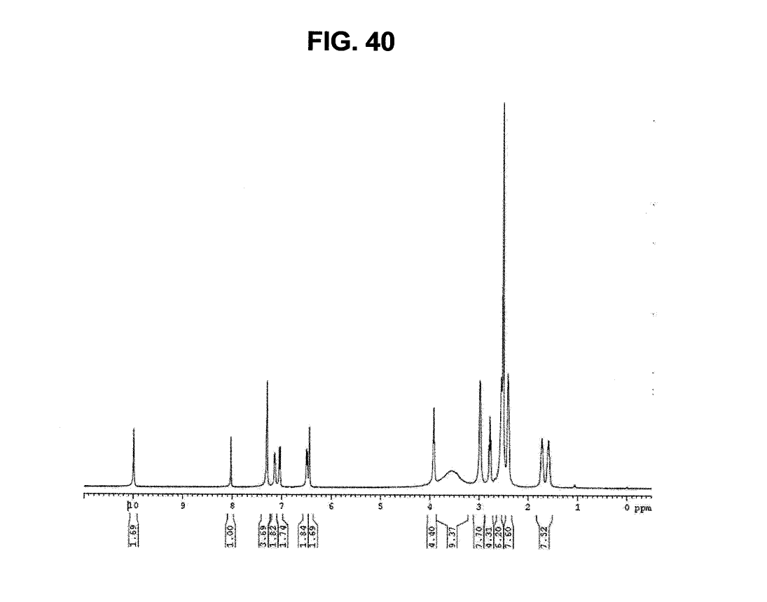

The peaks in FIG. 40 indicate that aripiprazole coexists with terephthalic acid, and that the ratio of hydrogen between the molecules is 2:1.

### Example 9

### Preparation of Aripiprazole-Terephthalic Acid Cocrystal by Grinding Process

500 mg of aripiprazole was mixed with 92.64 mg of terephthalic acid, and the mixture was placed in agate mortar, and ground uniformly with an agate pestle for 10 minutes. Alternatively, 0.2 mL of dimethyl sulfoxide was added to the mixture, which was then ground for 10 minutes and dried overnight in a hood.

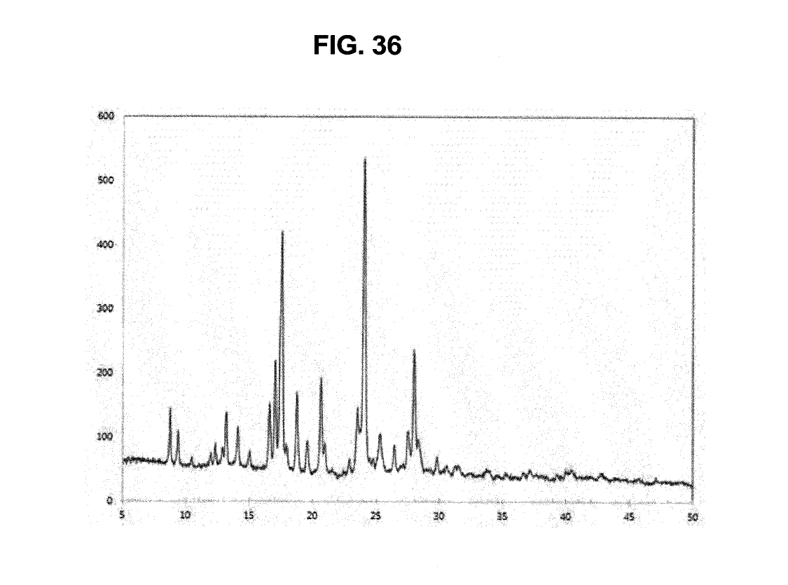

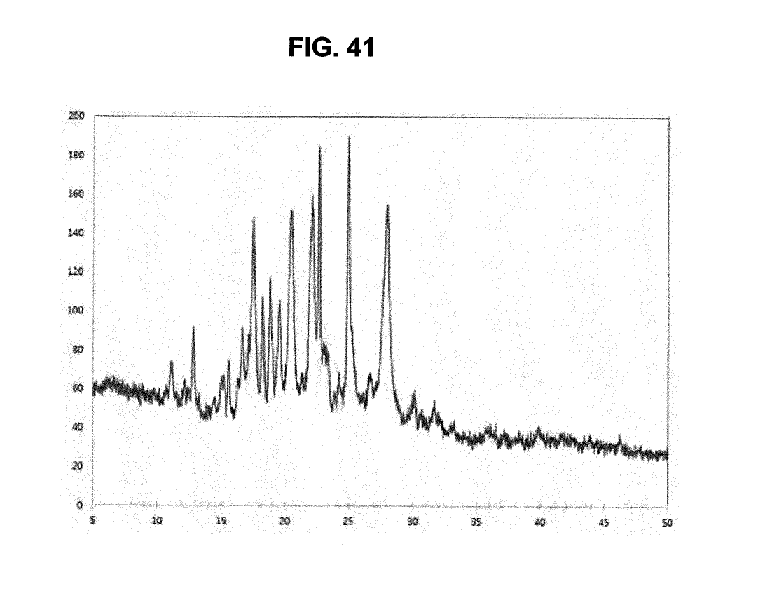

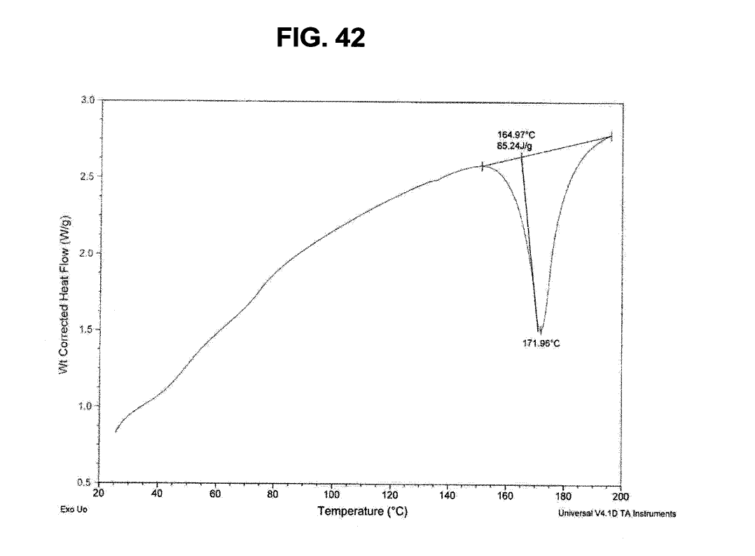

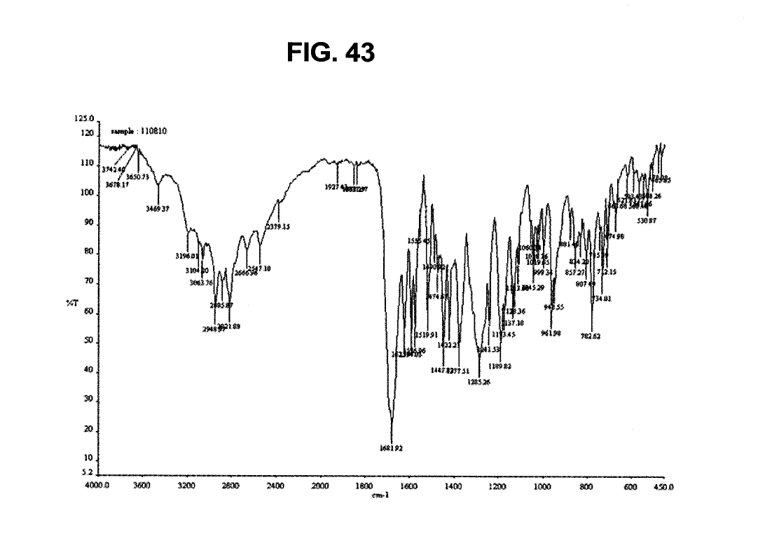

From a comparison between FIGS. 41 to 43 with FIGS. 36, 38 and 39, respectively, it can be seen that the cocrystal prepared in this Example is the same as the cocrystal prepared in Example 8. In the DSC data, the endothermic melting point of the sample prepared in Example 8 was higher than that of the sample of Example 9 due to sublimation of the solvent before melting.

Although the preferred embodiments of the present invention have been described with reference to the accompanying drawings, it should be understand that various modifications and changes are possible, without departing from the scope and spirit of the invention. Therefore, the scope of the present invention should not be defined by the described embodiments, but should be defined by the appended claims and equivalents thereof.

### MODE FOR INVENTION

According to the present invention, an aripiprazole-organic acid cocrystal comprises aripiprazole and an organic acid.

Furthermore, the organic acid is one or more of salicylic acid, adipic acid, benzenesulfonic acid, nicotinic acid, and terephthalic acid, and a molecular ratio of the aripiprazole to the organic acid is 1:1 or 2:1.

According to the present invention, a method for preparing an aripiprazole-organic acid cocrystal, comprises forming a cocrystal from aripiprazole and an organic acid by a solvent process and a grinding process.

Furthermore, the organic acid is one or more of salicylic acid, adipic acid, benzenesulfonic acid, nicotinic acid, and terephthalic acid, and a molecular ratio of the aripiprazole to the organic acid is 1:1 or 2:1.

Furthermore, the method comprises the steps of: dissolving the aripiprazole and the organic acid in a solvent to form a solution; and either crystallizing the solution under cold conditions at a temperature of 0˜10° C., or evaporating the solvent from the solution, thereby obtaining the cocrystal, wherein the solvent is one or a mixture of two or more selected from a group consisting of aprotic solvents, dimethylformamide, dimethyl sulfoxide, dimethylacetamide, acetonitrile, ether-based solvents, tetrahydrofuran, 1,4-dioxane, alcohol-based solvents, methanol, ethanol, isopropanol, propanol, isobutanol, n-butanol, t-butanol, ethylene glycol, dichloromethane, acetone, methyl ethyl ketone, and distilled water.

Furthermore, the method comprises grinding the aripiprazole and the organic acid in mortar, a grinder or a mill to obtain a crystal, or adding a solvent to the aripiprazole and the organic acid, followed by grinding and drying to obtain a crystal, wherein the solvent is one or a mixture of two or more selected from a group consisting of aprotic solvents, dimethylformamide, dimethyl sulfoxide, dimethylacetamide, acetonitrile, ether-based solvents, tetrahydrofuran, 1,4-dioxane, alcohol-based solvents, methanol, ethanol, isopropanol, propanol, isobutanol, n-butanol, t-butanol, ethylene glycol, dichloromethane, acetone, methyl ethyl ketone, and distilled water.

According to the present invention, a formulation or composition which contains a aripiprazole-organic acid cocrystal prepared according to the above-mentioned method, and which is a pharmaceutically acceptable oral, external, transdermal, transmucosal, injection or inhalation formulation of the cocrystal, and is one or more of tablet, coated tablet, soft capsule, powder, cream, ointment, patch, plaster, injection, aerogel and inhalation formulations.

### INDUSTRIAL APPLICABILITY

The present invention is a pharmaceutically acceptable oral, external, transdermal, transmucosal, injection or inhalation formulation.

1. An aripiprazole-organic acid cocrystal comprising aripiprazole and an organic acid.

2. The aripiprazole-organic acid cocrystal of claim 1, wherein the organic acid is one or more of salicylic acid, adipic acid, benzenesulfonic acid, nicotinic acid, and terephthalic acid, and a molecular ratio of the aripiprazole to the organic acid is 1:1 or 2:1.

3. A method for preparing an aripiprazole-organic acid cocrystal, comprising forming a cocrystal from aripiprazole and an organic acid by a solvent process and a grinding process.

4. The method of claim 3, wherein the organic acid is one or more of salicylic acid, adipic acid, benzenesulfonic acid, nicotinic acid, and terephthalic acid, and a molecular ratio of the aripiprazole to the organic acid is 1:1 or 2:1.

5. The method of claim 3, wherein the method comprises the steps of: dissolving the aripiprazole and the organic acid in a solvent to form a solution; and
either crystallizing the solution under cold conditions at a temperature of 0˜10° C., or evaporating the solvent from the solution, thereby obtaining the cocrystal, wherein the solvent is one or a mixture of two or more selected from a group consisting of aprotic solvents, dimethylformamide, dimethyl sulfoxide, dimethylacetamide, acetonitrile, ether-based solvents, tetrahydrofuran, 1,4-dioxane, alcohol-based solvents, methanol, ethanol, isopropanol, propanol, isobutanol, n-butanol, t-butanol, ethylene glycol, dichloromethane, acetone, methyl ethyl ketone, and distilled water.

6. The method of claim 3, wherein the method comprises grinding the aripiprazole and the organic acid in mortar, a grinder or a mill to obtain a crystal, or adding a solvent to the aripiprazole and the organic acid, followed by grinding and drying to obtain a crystal, wherein the solvent is one or a mixture of two or more selected from a group consisting of aprotic solvents, dimethylformamide, dimethyl sulfoxide, dimethylacetamide, acetonitrile, ether-based solvents, tetrahydrofuran, 1,4-dioxane, alcohol-based solvents, methanol, ethanol, isopropanol, propanol, isobutanol, n-butanol, t-butanol, ethylene glycol, dichloromethane, acetone, methyl ethyl ketone, and distilled water.

7. A formulation or composition which contains a aripiprazole-organic acid cocrystal prepared according to the method of claim 3, and which is a pharmaceutically acceptable oral, external, transdermal, transmucosal, injection or inhalation formulation of the cocrystal, and is one or more of tablet, coated tablet, soft capsule, powder, cream, ointment, patch, plaster, injection, aerogel and inhalation formulations.

8. The method of claim 4, wherein the method comprises the steps of: dissolving the aripiprazole and the organic acid in a solvent to form a solution; and either crystallizing the solution under cold conditions at a temperature of 0˜10° C., or evaporating the solvent from the solution, thereby obtaining the cocrystal, wherein the solvent is one or a mixture of two or more selected from a group consisting of aprotic solvents, dimethylformamide, dimethyl sulfoxide, dimethylacetamide, acetonitrile, ether-based solvents, tetrahydrofuran, 1,4-dioxane, alcohol-based solvents, methanol, ethanol, isopropanol, propanol, isobutanol, n-butanol, t-butanol, ethylene glycol, dichloromethane, acetone, methyl ethyl ketone, and distilled water.

9. The method of claim 4, wherein the method comprises grinding the aripiprazole and the organic acid in mortar, a grinder or a mill to obtain a crystal, or adding a solvent to the aripiprazole and the organic acid, followed by grinding and drying to obtain a crystal, wherein the solvent is one or a mixture of two or more selected from a group consisting of aprotic solvents, dimethylformamide, dimethyl sulfoxide, dimethylacetamide, acetonitrile, ether-based solvents, tetrahydrofuran, 1,4-dioxane, alcohol-based solvents, methanol, ethanol, isopropanol, propanol, isobutanol, n-butanol, t-butanol, ethylene glycol, dichloromethane, acetone, methyl ethyl ketone, and distilled water.

10. A formulation or composition which contains a aripiprazole-organic acid cocrystal prepared according to the method of claim 4, and which is a pharmaceutically acceptable oral, external, transdermal, transmucosal, injection or inhalation formulation of the cocrystal, and is one or more of tablet, coated tablet, soft capsule, powder, cream, ointment, patch, plaster, injection, aerogel and inhalation formulations.

11. A formulation or composition which contains a aripiprazole-organic acid cocrystal prepared according to the method of claim 5, and which is a pharmaceutically acceptable oral, external, transdermal, transmucosal, injection or inhalation formulation of the cocrystal, and is one or more of tablet, coated tablet, soft capsule, powder, cream, ointment, patch, plaster, injection, aerogel and inhalation formulations.

12. A formulation or composition which contains a aripiprazole-organic acid cocrystal prepared according to the method of claim 6, and which is a pharmaceutically acceptable oral, external, transdermal, transmucosal, injection or inhalation formulation of the cocrystal, and is one or more of tablet, coated tablet, soft capsule, powder, cream, ointment, patch, plaster, injection, aerogel and inhalation formulations.

13. A formulation or composition which contains a aripiprazole-organic acid cocrystal prepared according to the method of claim 8, and which is a pharmaceutically acceptable oral, external, transdermal, transmucosal, injection or inhalation formulation of the cocrystal, and is one or more of tablet, coated tablet, soft capsule, powder, cream, ointment, patch, plaster, injection, aerogel and inhalation formulations.

14. A formulation or composition which contains a aripiprazole-organic acid cocrystal prepared according to the method of claim 9, and which is a pharmaceutically acceptable oral, external, transdermal, transmucosal, injection or inhalation formulation of the cocrystal, and is one or more of tablet, coated tablet, soft capsule, powder, cream, ointment, patch, plaster, injection, aerogel and inhalation formulations.
